(19) United States
(12) Patent Application Publication
(10) Pub. No.: US 2024/0425855 A1
(43) Pub. Date: Dec. 26, 2024

(54) NUCLEIC ACID-CONTROLLED CATALYTIC RNAS FOR TRIGGER-RESPONSIVE REGULATION

(71) Applicant: Hoffmann-La Roche Inc., Little Falls, NJ (US)

(72) Inventors: Simon AUSLAENDER, Wolfratshausen (DE); Ignazio GERACI, Penzberg (DE)

(73) Assignee: Hoffmann-La Roche Inc., Little Falls, NJ (US)

(21) Appl. No.: 18/679,965

(22) Filed: May 31, 2024

Related U.S. Application Data
(63) Continuation of application No. PCT/EP2022/083596, filed on Nov. 29, 2022.

(30) Foreign Application Priority Data
Dec. 1, 2021 (EP) 21211547.1

(51) Int. Cl. C12N 15/113; C12N 15/10

(52) U.S. Cl. CPC C12N 15/113; C12N 15/1093; C12N 2310/121; C12N 2310/3231; C12N 2310/3519; C12N 2330/31

(57) ABSTRACT

Herein is reported a composition comprising a pair of a first nucleic acid and a second nucleic acid, wherein the first nucleic acid comprises in the order a first part of a stem nucleic acid sequence, a cleavage site, a first stem loop nucleic acid sequences which 5′- and 3′-parts form a duplex, a first part of a catalytic core sequence, a second stem loop nucleic acid sequences which 5′- and 3′-parts form a duplex, a second part of the catalytic core sequence, and a second part of the stem nucleic acid sequence, which is complementary to the first part of the stem nucleic acid sequence and, thus, form a duplex therewith, wherein the second nucleic acid is complementary to at least a part of the first or the second part of the stem nucleic acid sequence, wherein binding of the second nucleic acid to the first nucleic acid results in a conformational change of the first nucleic acid, which is at least one of the dissociation of the first part of the first stem sequence from the second part of the stem sequence and the hybridization of one of the parts with the second nucleic acid, or the dissociation of loop I and loop II resulting in an inactivation of the catalytic activity, or the association of loop I and loop II resulting in an activation of the catalytic activity.

The current invention is in the field of catalytic RNAs. More precisely, a self-cleaving or bond-forming catalytic RNA, e.g. a ribozyme, is combined with a nucleic acid binding site and introduced into the 3′UTR of a coding nucleic acid to control the expression of said coding nucleic acid depending on the presence of a nucleic acid sequence binding to the catalytic RNA.

### BACKGROUND OF THE INVENTION

Ribozymes are self-cleaving RNA molecules that can be engineered in a way to enable trigger-responsive control of self-cleaving activity. When embedded into 5′ or 3′UTR of mammalian mRNAs, those trigger-responsive ribozymes have been used for gene regulation purposes. A challenge is to engineer trigger-responsive ribozymes with low leakiness in the OFF state and high expression level in the ON state. Most of the small molecule-responsive ribozymes enable gene expression fold changes of 2-10, e.g. responsive to small molecules like theophylline, tetracycline, guanine and others.

Recently, morpholino-controlled ribozymes have been reported that demonstrated more than 100-fold difference of gene expression in the ON versus OFF state (Zhong, G., et al., Nat Biotechnol. 2020 38 (2020) 169-175).

Mudiyanselage et al. (Methods 161 (2019) 24-34) reviewed second-generation fluorogenic RNA-based sensors. They outlined that aptamers can bind and activate the fluorescence of different classes of fluorophores, and as a result, target detection at different wavelengths is now feasible. For example, Spinach, also known as the RNA mimic of green fluorescent proteins, is a 98-nucleotide long RNA aptamer that can specifically bind and switch on the fluorescence of a chromophore, 3,5-difluoro-4-hydroxy benzylidene imidazolinone (DFHBI).

Felletti and Hartig (Wiley Interdisciplinary Reviews: RNA, 8 (2017) 2-e1395) reviewed ligand-dependent ribozymes. They summarized the occurrence of ligand-dependent ribozymes in nature and the many examples realized by researchers that engineered ligand-dependent catalytic RNA motifs and methods for obtaining ligand dependency.

However, most of the currently known and applied ribozyme switches have a low dynamic range, high leakiness and/or non-optimal nature of the trigger, e.g. not suitable for human gene therapy due to non-favorable safety, toxicity or biodistribution properties.

The development of a targeted integration Chinese hamster ovary host directly targeting either one or two vectors simultaneously to a single locus using the Cre/Lox recombinase-mediated cassette exchange system has been reported by Ng et al. (Biotechnol. Prog. 37 (2021) 1-10).

A reversible RNA on-switch that controls gene expression of AAV-delivered therapeutics in vivo has been reported by Zhong Guocai (Nat. Biotechnol. 38 (2019) 169-175.

A general design strategy for protein-responsive riboswitches in mammalian cells and the engineering of a ribozyme cleavage-induced split fluorescent aptamer complementation assay have been reported by Simon Auslaender et al. (Nat. Meth. 11 (2014) 1154-1160; Nucl. Acids Res. 44 (2016) 1-7).

WO 2021/076563 discloses RNA switches that are based on modified hammerhead ribozymes with improved activities, wherein the most efficient regulators are octa-guanidine dendrimer-coupled morpholino binding to the internal part of the RNA switch downstream of the cleavage site.

### SUMMARY OF THE INVENTION

The current invention is based, at least in part, on the finding that placing a self-cleaving or bond-forming catalytic RNA, e.g. a ribozyme, in combination with a nucleic acid binding site, e.g. in the stem region of the catalytic RNA, in the 3′UTR of a coding nucleic acid, e.g. of a (selection) marker gene such as eGFP, but prior to the polyA signal sequence can be used to control the expression of said coding nucleic acid depending on the presence of a nucleic acid sequence binding to the nucleic acid sequence binding site of the catalytic RNA.

The nucleic acid according to the invention allows for different applications.

In one aspect of the invention, the nucleic acid according to the invention is used for the selective activation or inactivation of the expression of an operably linked coding nucleic acid. The coding nucleic acid is selected, in one embodiment, from the group comprising selection marker encoding nucleic acids, non-therapeutic protein encoding nucleic acids, therapeutic nucleic acids, and therapeutic protein encoding nucleic acids.

In one aspect of the invention, the nucleic acid according to the invention is used for the screening and selection of ribozyme-nucleic acid sequence pairs wherein the nucleic acid sequence activates or inhibits the catalytic activity of the ribozyme.

Thus, one aspect according to the current invention is a method for selecting a ribozyme based on the leakiness or tightness of the regulating properties of the regulating nucleic acid.

Thus, one aspect of the current invention is a first nucleic acid or a second nucleic acid or a pair of a first nucleic acid and a second nucleic acid,
wherein the first nucleic acid comprises in the following sequence (in 5′- to -3′- or 3′- to -5′-direction)
   a first part of a stem nucleic acid sequence,
   a cleavage site,
   a first stem loop nucleic acid sequences which 5′- and 3′-parts can form/form a duplex (loop I),
   a first part of a catalytic core sequence,
   a second stem loop nucleic acid sequences which 5′- and 3′-parts can form/form a duplex (loop II),
   a second part of the catalytic core sequence, and
   a second part of the stem nucleic acid sequence, which is complementary to the first part of the stem nucleic acid sequence (and, thus, can form/forms a duplex therewith),
wherein the second nucleic acid is complementary to at least a part of the first or the second part of the stem nucleic acid sequence,
wherein binding of the second nucleic acid to the first nucleic acid results in a conformational change of the first nucleic acid, which is at least one of the following
   the dissociation of the first part of the first stem sequence from the second part of the stem sequence and the hybridization of one of the parts with the second nucleic acid,
   the dissociation of loop I and loop II resulting in an inactivation of the catalytic activity,
   the association of loop I and loop II resulting in an activation of the catalytic activity.

In certain embodiments of all aspects and embodiments of the invention, the first nucleic acid is a hammerhead ribozyme and the stem is loop III of the hammerhead ribozyme.

In certain embodiments of all aspects and embodiments of the invention, the second nucleic acid is a LNA (locked nucleic acid).

A further aspect of the current invention is a fusion nucleic acid comprising
a coding nucleic acid encoding a selectable marker or a therapeutic protein or a regulatory compound, in one preferred embodiment a fluorescent protein or an antibody or a regulatory protein,
a nucleic acid comprising the first nucleic acid according to the invention, and
a polyadenylation signal sequence that is operably linked to the coding nucleic acid (in case the second nucleic acid sequence is catalytically inactive).

A further aspect of the current invention is a mammalian cell comprising the first nucleic acid according to the invention or the fusion nucleic acid according to the invention.

A further aspect of the current invention is a method for selecting a pair of a first nucleic acid and a second nucleic acid according to the invention comprising the following steps:
providing a library of fusion nucleic acids according to the invention wherein all or a part of the stem nucleic acid in the first nucleic acid according to the invention is randomized (e.g. by error prone PCR or synthesis or mutation, etc.),
integrating the members of the fusion nucleic acid library into mammalian cells by targeted integration (by using targeted integration only a single member of the library is introduced into the genome of one cell even if multiple members are provided/present),
single depositing the cells after the integration to generate a single cell library (wherein each member of the single cell library comprises one member of the fusion nucleic acid library),
determining the members of the single cell library in which the catalytic activity of the first nucleic acid can be changed by the addition of a second nucleic acid (e.g. a second nucleic acid library) by determining the expression level of the coding nucleic acid,
selecting a member of the single cell library wherein the difference of expression level of the coding nucleic acid in the absence and the presence of the second nucleic acid is larger than for other cells of the single cell library.

In certain embodiments of all aspects and embodiments of the invention, the targeted integration is by recombinase mediated cassette exchange (RMCE).

In certain embodiments of all aspects and embodiments of the invention, the targeted integration is by double recombinase mediated cassette exchange (double RMCE).

Using double RMCE allows the targeted integration of the fusion nucleic acid according to the invention and a further nucleic acid comprising a (second different) selectable marker. By using two different integrated encoding nucleic acids, e.g. the expression level of the (further second) selectable marker can be used for the normalization of the expression level of the fusion nucleic acid (to ensure comparability of the results; normalization) and also for the deselection of cells with not correctly integrated fusion nucleic acid.

In certain embodiments of all aspects and embodiments of the invention, the elements of the first nucleic acid are recited in 5′-to-3′ direction.

In certain embodiments of all aspects and embodiments of the invention, one aspect is a pair of a first nucleic acid according to the invention and a second nucleic acid according to the invention.

In certain embodiments of all aspects and embodiments of the invention, the first nucleic acid is in the 3′-UTR of a coding sequence.

In certain embodiments of all aspects and embodiments of the invention, the first nucleic acid is after a coding sequence and before a poly A signal sequence.

In certain embodiments of all aspects and embodiments of the invention, the coding sequence is in an expression cassette.

In certain embodiments of all aspects and embodiments of the invention, the coding sequence is 5′ to the first nucleic acid and a poly A signal sequence is 3′ to the first nucleic acid.

In certain embodiments of all aspects and embodiments of the invention, the coding sequence and the poly A signal sequence are operably linked.

In certain embodiments of all aspects and embodiments of the invention, the second nucleic acid comprises a first part that is complementary to at least a part of the first or the second part of the stem nucleic acid sequence and a second part that is complementary to the nucleic acid sequence 5′ to the first part of the stem nucleic acid sequence in case the second nucleic acid is complementary to at least a part of the first part of the stem nucleic acid sequence or that is complementary to the nucleic acid sequence 3′ to the second part of the stem nucleic acid sequence in case the second nucleic acid is complementary to at least a part of the second part of the stem nucleic acid sequence.

In one preferred embodiment of all aspects and embodiments of the invention, the second nucleic acid is complementary to the second part of the stem nucleic acid sequence.

In certain embodiments of all aspects and embodiments of the invention, the second nucleic acid comprises or consists of a first part that is complementary at least a part of the stem nucleic acid sequence and a second part that is not complementary to the first nucleic acid sequence.

In certain embodiments of all aspects and embodiments of the invention, the second nucleic acid is not complementary to the first nucleic acid outside the stem nucleic acid sequence.

In one preferred embodiment of all aspects and embodiments of the invention, the second nucleic acid is LNA.

In certain embodiments of all aspects and embodiments of the invention, the second nucleic acid has a length of 8 to 21 nucleotides. In certain embodiments of all aspects and embodiments of the invention, the second nucleic acid has a length of 9 to 20 nucleotides. In one preferred embodiment of all aspects and embodiments of the invention, the second nucleic acid has a length of 11 to 18 nucleotides.

In certain embodiments of all aspects and embodiments of the invention, the second nucleic acid has a first part of 8-13 nucleotides that are complementary to the stem nucleic acid sequence of the first nucleic acid and a second part of 13 to 8 nucleotides that are complementary to a nucleic acid sequence 5′ or 3′ to the stem nucleic acid sequences of the first nucleic acid, i.e. which is not part of the first nucleic acid and is outside of the first nucleic acid. In one preferred embodiment of all aspects and embodiments of the invention, the second nucleic acid has a first part of about 11 nucleotides and a second part of about 7 nucleotides.

In certain embodiments of all aspects and embodiments of the invention, wherein every nucleotide of the second nucleic acid has a phosphorothioate backbone.

In certain embodiments of all aspects and embodiments of the invention, the first nucleic acid comprises
as first part of a stem nucleic acid sequence the sequence of CCU CGU (SEQ ID NO: 01),
as cleavage site the sequence of CC (SEQ ID NO: 02),
as first stem loop nucleic acid sequences, which 5′- and 3′-parts form a duplex, the sequence of GGG GCU GGA CCG CCC C (SEQ ID NO: 03),
as first part of the catalytic core sequence the sequence of GCU GAC GA (SEQ ID NO: 04),
as second stem loop nucleic acid sequences, which 5′- and 3′-parts form a duplex, the sequence of GGC CCG CGG AGG GCC (SEQ ID NO: 05),
as second part of the catalytic core sequence the sequence of GAA (SEQ ID NO: 06),
as second part of the stem nucleic acid sequence, which is complementary to the first part of the stem nucleic acid sequence, and forms a duplex therewith the sequence of ACG AGG AGG (SEQ ID NO: 08).

In certain embodiments of all aspects and embodiments of the invention, the first part of the stem nucleic acid sequence has the sequence of CTG AGG GTA GT (SEQ ID NO: 09) and the second part of the stem nucleic acid sequence has the sequence of ACT ACC CTC AG (SEQ ID NO: 10).

In certain embodiments of all aspects and embodiments of the invention, the second nucleic acid has the sequence of is ATT GTG CCT GAG GGT AGT (SEQ ID NO: 13).

In certain embodiments of all aspects and embodiments of the invention, wherein the second nucleic acid comprises more nucleotides as those nucleotides complementary to the first or second part of the first nucleic acid, but which are not complementary to other parts of the first nucleic acid.

In certain embodiments of all aspects and embodiments of the invention, the first nucleic acid is within the 3′-UTR of an expression cassette between the coding sequence and the poly A signal sequence.

In addition to the various aspects and embodiments depicted and claimed, the disclosed subject matter is also directed to other aspects and embodiments having other combinations of the features disclosed and claimed herein. As such, the particular features presented herein, especially presented as aspects or embodiments, can be combined with each other in other manners within the scope of the disclosed subject matter such that the disclosed subject matter includes any suitable combination of the features disclosed herein. The description of specific embodiments of the disclosed subject matter has been presented for purposes of illustration and description. It is not intended to be exhaustive or to limit the disclosed subject matter to those embodiments disclosed.

### DETAILED DESCRIPTION OF THE INVENTION

The current invention is based, at least in part, on the finding that placing a self-cleaving or bond-forming catalytic RNA, e.g. a ribozyme, in combination with a nucleic acid binding site, e.g. in the stem region of the catalytic RNA, in the 3′UTR of a coding nucleic acid, e.g. of a (selection) marker gene such as eGFP, but prior to the polyA signal sequence can be used to control the expression of said coding nucleic acid depending on the presence of a nucleic acid sequence binding to the nucleic acid sequence binding site of the catalytic RNA.

RNA-based gene switches are highly desirable for gene therapy, especially for the conditional activation of the introduced therapeutic transgene. Their advantages are, amongst other things the small size of about 100 nucleotides (nt), the absence of immunogenicity (no protein is involved) and the low complexity (only RNA is required).

The current invention is based, at least in part, on the finding that the nucleic acid-responsive ribozyme design according to the invention is generally applicable. The switching part in the nucleic acid-responsive ribozyme design according to the invention can be easily modified.

The nucleic acid-responsive ribozyme design according to the invention, i.e. the design of specific nucleic acid-ribozyme pairs, allows the use of any nucleic acid, such as, e.g., LNA, of interest and connecting it to the control of gene expression. LNAs are already in clinical trials and are suitable for being employed in in vivo gene therapy.

### DEFINITIONS AND GENERAL DESCRIPTIONS

### General Definitions

Useful methods and techniques for carrying out the current invention are described in e.g. Ausubel, F. M. (ed.), Current Protocols in Molecular Biology, Volumes I to III (1997): Glover, N. D., and Hames, B. D., ed., DNA Cloning: A Practical Approach, Volumes I and II (1985), Oxford University Press: Freshney, R. I. (ed.), Animal Cell Culture—a practical approach, IRL Press Limited (1986): Watson, J. D., et al., Recombinant DNA, Second Edition, CHSL Press (1992): Winnacker, E. L., From Genes to Clones: N.Y., VCH Publishers (1987): Celis, J., ed., Cell Biology, Second Edition, Academic Press (1998): Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA: Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “comprising” also encompasses the term “consisting of”.

Polymerases, for example, move from the 5′-end to the 3′-end of nucleic acids. Thus, the nucleotide sequence of a nucleic acid is written down in the same 5′-to-3′-direction from left to right. This is especially applied for coding nucleic acid sequences, e.g. within expression cassettes, to reflect the reading direction of the ribosome during the translation process. Thus, the 5′-end the beginning or front or the left end of a nucleic acid sequence and the 3′-end is the end or the right end of a nucleic acid sequence. The terms 5′-end and 3′-end are also used for the characterization of the orientation of coding sequences. Thus, the promoter that is operably linked to a coding sequence is 5′ to the coding sequence, i.e. in front of it.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader and a polypeptide, contiguous and in (reading) frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distance from the promoter. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

### Cell-Specific Definitions:

The term “cell clone” as used herein denotes a mammalian cell comprising an exogenous nucleotide sequence capable of expressing a polypeptide, i.e. a recombinant mammalian cell. Such recombinant mammalian cells are cells into which one or more exogenous nucleic acid(s) have been introduced, including the progeny of such cells. In certain embodiments, the cell clone is a mammalian cell comprising a nucleic acid encoding a heterologous polypeptide. Thus, the term “cell clone comprising a nucleic acid encoding a heterologous polypeptide” denotes recombinant mammalian cells comprising an exogenous nucleotide sequence integrated in the genome of the mammalian cell and capable of expressing the heterologous polypeptide. In certain embodiments, the cell clone is a mammalian cell comprising an exogenous nucleotide sequence integrated at a single site within a locus of the genome of the cell. In one preferred embodiment, the cell clone is a mammalian cell comprising an exogenous nucleotide sequence integrated at a single site within a locus of the genome of the cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

The term “recombinant cell” as used herein denotes a cell after genetic modification, such as, e.g., a cell expressing a heterologous polypeptide of interest and that can be used for the production of said heterologous polypeptide of interest at any scale. For example, “a cell clone” denotes a cell wherein the coding sequences for a heterologous polypeptide of interest have been introduced into the genome. For example, “a recombinant mammalian cell comprising an exogenous nucleotide sequence” that has been subjected to recombinase mediated cassette exchange (RMCE), whereby the coding sequences for a polypeptide of interest have been introduced into the genome of the host cell, is a specific “cell clone”.

A “cell clone” as used herein denotes a “transformed cell”. This includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated cell clone” denotes a cell clone, which has been separated from a component of its natural environment.

An “isolated nucleic acid” denotes a nucleic acid molecule that has been separated from a component of its natural environment.

### Targeted Integration:

One method for the generation of a recombinant mammalian cell clone to be used in the method according to the current invention is a recombinant cell clone generated by using targeted integration (TI) for the introduction of the fusion nucleic acid.

In targeted integration, site-specific recombination is employed for the introduction of an exogenous nucleic acid into a specific locus in the genome of a mammalian TI host cell to generate recombinant cell clones. This is an enzymatic process wherein a sequence at the site of integration in the genome is exchanged for the exogenous nucleic acid. One system used to effect such nucleic acid exchanges is the Cre-lox system. The enzyme catalyzing the exchange is the Cre recombinase. The sequence to be exchanged is defined by the position of two lox(P)-sites in the genome as well as in the exogenous nucleic acid. These lox(P)-sites are recognized by the Cre recombinase. Nothing more is required, i.e. no ATP etc. Originally, the Cre-lox system has been found in bacteriophage P1.

The Cre-lox system operates in different cell types, like mammals, plants, bacteria and yeast.

In certain embodiments, the fusion nucleic acid has been integrated into the mammalian TI host cell by single or double recombinase mediated cassette exchange (RMCE). Thereby a recombinant mammalian cell clone, such as a recombinant CHO cell clone, is obtained, in which the fusion nucleic acid has been integrated into the genome at a single locus.

The Cre-LoxP site-specific recombination system has been widely used in many biological experimental systems. Cre recombinase is a 38-kDa site-specific DNA recombinase that recognizes 34 bp LoxP sequences. Cre recombinase is derived from bacteriophage P1 and belongs to the tyrosine family site-specific recombinase. Cre recombinase can mediate both intra- and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre recombinase-mediated recombination will excise DNA sequences located between the two LoxP sequences as a covalently closed circle. If two LoxP sequences are placed in an inverted position on the same nucleotide sequence, Cre recombinase-mediated recombination will invert the orientation of the DNA sequences located between the two sequences. If two LoxP sequences are on two different DNA molecules and if one DNA molecule is circular, Cre recombinase-mediated recombination will result in integration of the circular DNA sequence.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

The term “matching RRSs” indicates that a recombination occurs between two RRSs. In certain embodiments, the two matching RRSs are the same.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

A “two-plasmid RMCE” strategy or “double RMCE” is employed in the method according to the current invention when using a two-vector combination, i.e. a first vector comprising the fusion nucleic acid according to the invention and a second vector comprising a further selectable marker. For example, but not by way of limitation, an integrated landing site could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

The two-plasmid RMCE strategy involves using three RRS sites to carry out two independent RMCEs simultaneously. Therefore, a landing site in the mammalian TI host cell using the two-plasmid RMCE strategy includes a third RRS site (RRS3) that has no cross activity with either the first RRS site (RRS1) or the second RRS site (RRS2). The two plasmids to be targeted require the same flanking RRS sites for efficient targeting, one plasmid (front) flanked by RRS1 and RRS3 and the other (back) by RRS3 and RRS2. In addition, two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in frame translation for the fusion protein, i.e. operable linkage. Only when both plasmids are correctly inserted, the full expression cassette of the selection marker will be assembled and, thus, rendering cells resistance to the respective selection agent.

Two-plasmid RMCE involves double recombination crossover events, catalyzed by a recombinase, between the two heterospecific RRSs within the target genomic locus and the donor DNA molecule. Two-plasmid RMCE is designed to introduce a copy of the DNA sequences from the front- and back-vector in combination into the pre-determined locus of a mammalian TI host cell's genome. RMCE can be implemented such that prokaryotic vector sequences are not introduced into the mammalian TI host cell's genome, thus, reducing and/or preventing unwanted triggering of host immune or defense mechanisms. The RMCE procedure can be repeated with multiple DNA sequences.

In certain embodiments, targeted integration is achieved by two RMCEs, wherein two different DNA sequences, each flanked by two heterospecific RRSs, are both integrated into a pre-determined site of the genome of a RRSs matching mammalian TI host cell. In certain embodiments, targeted integration is achieved by multiple RMCEs, wherein DNA sequences from multiple vectors, each integrated sequence being flanked by two heterospecific RRSs, are all integrated into a predetermined site of the genome of a mammalian TI host cell. In certain embodiments the selection marker can be partially encoded on the first the vector and partially encoded on the second vector such that only the correct integration of both by double RMCE allows for the expression of the selection marker.

An exemplary mammalian TI host cell that is suitable for use in a method according to the current invention is a CHO cell harboring a landing site integrated at a single site within a locus of its genome wherein the landing site comprises three heterospecific loxP sites for Cre recombinase mediated DNA recombination.

In this example, the heterospecific loxP sites are L3, LoxFas and 2L (see e.g. Lanza et al., Biotechnol. J. 7 (2012) 898-908: Wong et al., Nucleic Acids Res. 33 (2005) e147), whereby L3 and 2L flank the landing site at the 5′-end and 3′-end, respectively, and LoxFas is located between the L3 and 2L sites.

Such a configuration of the landing site as outlined in the previous paragraph allows for the simultaneous integration of two vectors, e.g. of a so called front vector harboring an L3 and a LoxFas site and a back vector harboring a LoxFas and an 2L site. The functional elements of a selection marker gene different from that present in the landing site can be distributed between both vectors: promoter and start codon can be located on the front vector whereas coding region and poly A signal are located on the back vector. Only correct recombinase-mediated integration of said nucleic acids from both vectors induces resistance against the respective selection agent.

Generally, a mammalian TI host cell is a mammalian cell comprising a landing site integrated within a locus of the genome of the mammalian cell, wherein the landing site comprises a first and a second recombination recognition sequence flanking at least one first selection marker, and a third recombination recognition sequence located between the first and the second recombination recognition sequence, and all the recombination recognition sequences are different.

An exogenous nucleotide sequence is a nucleotide sequence that does not originate from a specific cell but can be introduced into said cell by DNA delivery methods, such as, e.g., by transfection, electroporation, or transformation methods. In certain embodiments, a mammalian TI host cell comprises at least one landing site integrated at one or more integration sites in the mammalian cell's genome. In certain embodiments, the landing site is integrated at one or more integration sites within a specific locus of the genome of the mammalian cell.

In certain embodiments, the integrated landing site comprises at least one selection marker. In certain embodiments, the integrated landing site comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments, a selection marker is located between a first and a second RRS and the two flanking RRSs are different. In certain preferred embodiments, the first flanking RRS is a LoxP L3 sequence and the second flanking RRS is a LoxP 2L sequence. In certain embodiments, a LoxP L3 sequence is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientations.

### Host Cells:

Any mammalian host cell line that is adapted to grow in suspension can be used to generate recombinant cell clones that can be processed in the method according to the current invention.

Examples of useful mammalian host cell lines are human amniocyte cells (e.g. CAP-T cells as described in Woelfel, J. et al., BMC Proc. 5 (2011) P133): monkey kidney CV1 line transformed by SV40 (COS-7): human embryonic kidney line (HEK293 or HEK293T cells as described, e.g., in Graham, F. L. et al., J. Gen Virol. 36 (1977) 59-74): baby hamster kidney cells (BHK): mouse sertoli cells (TM4 cells as described, e.g., in Mather, J. P., Biol. Reprod. 23 (1980) 243-252): monkey kidney cells (CV1): African green monkey kidney cells (VERO-76): human cervical carcinoma cells (HELA): canine kidney cells (MDCK: buffalo rat liver cells (BRL 3A): human lung cells (W138): human liver cells (Hep G2): mouse mammary tumor (MMT 060562); TRI cells (as described, e.g., in Mather, J. P. et al., Annals N.Y. Acad. Sci. 383 (1982) 44-68): MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P, and Wu, A. M., Methods in Molecular Biology, Vol. 248, Lo, B. K. C. (ed.), Humana Press, Totowa, NJ (2004), pp. 255-268.

In certain embodiments, the mammalian host cell is, e.g., a Chinese Hamster Ovary (CHO) cell (e.g. CHO K1, CHO DG44, etc.), a Human Embryonic Kidney (HEK) cell, a lymphoid cell (e.g., Y0, NS0, Sp2/0 cell), or a human amniocyte cells (e.g. CAP-T, etc.). In one preferred embodiment, the mammalian (host) cell is a CHO cell. Thus, likewise the cell clone is a CHO cell.

With respect to TI, any known or future mammalian host cell suitable for TI comprising a landing site as described herein integrated at a single site within a locus of the genome can be used in the current invention. Such a cell is denoted as a mammalian TI host cell. In certain embodiments, the mammalian TI host cell is a hamster cell, a human cell, a rat cell, or a mouse cell comprising a landing site as described herein. In one preferred embodiment, the mammalian TI host cell is a CHO cell. In certain embodiments, the mammalian TI host cell is a Chinese hamster ovary (CHO) cell, a CHO K1 cell, a CHO K1SV cell, a CHO DG44 cell, a CHO DUKXB-11 cell, a CHO K1S cell, or a CHO K1M cell comprising a landing site as described herein integrated at a single site within a locus of the genome.

In certain embodiments, a mammalian TI host cell comprises an integrated landing site, wherein the landing site comprises one or more recombination recognition sequence (RRS). The RRS can be recognized by a recombinase, for example, a Cre recombinase, an FLP recombinase, a Bxb1 integrase, or a φC31 integrase. The RRS can be selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

If multiple RRSs have to be present, the selection of each of the sequences is dependent on the other insofar as non-identical RRSs are chosen.

### Combinatorial Libraries:

RMCE-based targeted integration can be used for generating combinatorial expression libraries in CHO cells in which each cell comprises exactly one fusion nucleic acid.

Thus, an exemplary RMCE-based method for generating a recombinant cell library comprising a library of fusion nucleic acids according to the invention comprises:
a) providing a targeted integration host cell comprising an exogenous nucleotide sequence integrated at a site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a library of first vectors each comprising two recombination recognition sequences that are matching the first and the third recombination recognition sequence on the integrated exogenous nucleotide sequence, wherein the two recombination recognition sequences are flanking the randomized fusion nucleic acid and at least (a part of) one second selection marker, and a single second vector comprising two recombination recognition sequences that are matching the second and the third recombination recognition sequence on the integrated exogenous nucleotide sequence, wherein the two recombination recognition sequences are flanking the further selection marker nucleic acid sequence;
c) introducing i) either simultaneously with the libraries of first vectors of b); or ii) sequentially thereafter one or more recombinases,
wherein the one or more recombinases recognize the recombination recognition sequences of the first and second vectors: (and optionally wherein the one or more recombinases perform two recombinase mediated cassette exchanges;)
and
d) selecting for recombinant host cells expressing the second and/or further selection marker,
thereby preparing a recombinant fusion nucleic acid cell library.

For the generation of a library of recombinant mammalian cells, such as, e.g., CHO cells, wherein each cell expresses a single member of the fusion nucleic acid library, a library of front plasmids comprising the different members of the fusion nucleic acid library and a single back plasmids comprising the further selection marker are mixed and transfected into the TI host. Mediated by Cre recombinase, the front plasmids are randomly paired with the back plasmid at the target locus. Subsequently, the pool of stably transfected cells is selected with Selectable Marker 2 (positive selection) and 3 (negative selection) and the further selection marker.

An expression library of cells obtained thereby is subjected to single-cell cloning by methods such as limiting dilution, cell sorting, or cell printing. The resulting single cells or their clonal descendants can be screened for the desired activation/inactivation by the second nucleic acid.

### Specific Embodiments of the Invention

Ribozymes are a class of RNA molecules capable of catalyzing the break of or the formation of a covalent bond within a nucleic acid molecule. Usually ribozymes are self-cleaving.

At least nine classes of naturally occurring small self-cleaving ribozymes have been described so far: the hammerhead, hairpin, human Hepatitis-δ, Varkud-satellite, GlmS, twister, twister sister, hatchet and pistol ribozymes (de la Pena et al., Molecules, 22 (2017) 78).

For example, the class of hammerhead ribozymes comprise a core catalytic site surrounded by three stems and tertiary loop-loop interactions, which are required for high self-cleavage activity of the ribozyme function (Martick and Scott, Cell, 126 (2006) 309-320).

However, a single nucleotide swap in the catalytic core (A→G) inactivates the self-cleaving activity of a hammerhead ribozyme. The difference in GFP expression determined by FACS is shown in FIG. 1 and a sketch of the respective construct in FIG. 2.

With the method according to the current invention it is possible to select variant ribozymes with minimal leakiness, i.e. expression, in the absence of an external stimulus, by selecting cells showing the highest expression of the marker gene in the presence of the stimulus and the lowest expression of the marker gene in the absence of the stimulus, and vice versa. Thus, the fold-inactivation or fold-activation change can be used as a selection criterion. This is shown in FIG. 3.

A “riboswitch” is a regulatory ribonucleic acid sequence, i.e. a part of an mRNA molecule, that regulates the translation of said mRNA molecule. Thus, it is a cis-acting element. The catalytic activity of a riboswitch is dependent on the binding of an effector molecule. Thereby the three-dimensional structure of the riboswitch is changed from, e.g., the inactive into the active form. A riboswitch is composed of three domains: an aptamer domain, a switching sequence and an expression platform. In the absence of the effector molecule, the expression platform incorporates the switching sequence into an anti-terminator stem-loop (AT) and transcription proceeds through the coding region of the mRNA. In the presence of the effector molecule, the switching sequence is incorporated into the aptamer domain, and the expression platform folds into a terminator stem-loop (T), causing transcription to abort. That is, the expression platform can switch between two different secondary structures, whereof one is transcriptionally active and one is transcriptionally inactive, in response to effector molecule binding. (https://www.nature.com/scitable/topicpage/riboswitches-a-common-rna-regulatory-element-14262702/)

Self-cleaving RNA molecules are termed “ribozymes”. A specific class of ribozymes are “hammerhead ribozymes” (HHRs). Their cleavage activity is strongly influenced by tertiary loop-loop interactions, i.e. the correct tertiary structure.

Herein as an example a type III HHR-derived modular ribozyme scaffold denoted as “Env140” as reported by Auslaender et al. (Nature Meth. 11 (2014) 1154) has been used as an example. However, based on the teaching provided herein any other self-cleaving ribozyme can be used in the method according to the invention.

The combinations of HHRs with RNA aptamers responsive to effector molecules render the HHR's self-cleavage activity ligand-dependent. HHRs fold into a distinct tertiary structure composed of a three-way junction where stem loops I/II form a specific tertiary interaction required for efficient self-cleavage. Although the catalytic region is highly conserved, the nucleotide composition of the stem loops differs within individual HHR species, indicating that there are many methods to form the required loop-loop interaction that facilitates folding into an active ribozyme conformation.

The method according to the current invention is exemplified first in the following using the K4/K19 tetracycline-responsive ribozyme-switch generated by Beilstein et al. (ACS Synth. Biol., 15 (2015) 526-534). This is presented solely as an illustration of the method according to the invention and shall not be construed as limitation thereof. The true scope is set forth in the appended claims.

Whilst the output measurement was different (Luciferase vs GFP) a 1.9× fold-increase at 50 μM tetracycline concentration was achieved (FIG. 4 and FIG. 5).

Thus, it is shown that the introduction of a trigger-inactivated ribozyme in the 3″ UTR between the coding sequence and the poly A signal sequence can be efficiently used to induce off-switching of the expression of the marker gene in stably transfected cells.

Another ribozyme is Env140. This ribozyme has been described by Auslaender et al. (Nucl. Acids Res., 44 (2016) e94) expressly incorporated herein by reference. FIG. 2A in combination with FIG. 3A of Auslaender et al. show the structure of Env140. These are reproduced in FIG. 6.

Env140 consists of three stem-loop structures (I, II, III) that form a three-way junction surrounding the conserved catalytic core sequence. Tertiary loop I contacts tertiary loop II contacts are indicated by red lines; the red arrowhead points to the cleavage site; nucleotides in italics highlight the A-to-G inactivating mutation; nucleotides in blue represent a ribosome-binding site (RBS).

The 5′-to-3′ sequence of Env140 is as follows:
a first part of a stem nucleic acid sequence:

(SEQ ID NO: 01)
CCU CGU,

a cleavage site,
   C(!)C (SEQ ID NO: 02)
a first stem loop nucleic acid sequences which 5′- and 3′-parts can form/form a duplex (loop I),

(SEQ ID NO: 03)
GGG GCU GGA CCG CCC C

a first part of a catalytic core sequence,

(SEQ ID NO: 04)
GCU GAC GA

a second stem loop nucleic acid sequences which 5′- and 3′-parts can form/form a duplex (loop II),

(SEQ ID NO: 05)
GGC CCG CGG AGG GCC

a second part of the catalytic core sequence,
   GAA (active) (SEQ ID NO: 06)
   GAG (inactive) (SEQ ID NO: 07)
a second part of the stem nucleic acid sequence, which is complementary to the first part of the stem nucleic acid sequence (and, thus, can form/forms a duplex therewith),

(SEQ ID NO: 08)
ACG AGG AGG.

This corresponds to the sequence of the first nucleic acid except for that either the first part of the stem nucleic acid sequence or the second part of the stem nucleic acid sequence is modified to hybridize to an effector nucleic acid, such as, e.g., an effector LNA.

A sketch of an exemplary first nucleic acid according to the invention is shown in FIG. 7.

The effector nucleic acid hybridizes exactly with its 3′-terminal nucleotide to the 5′-terminal nucleotide of the second part of the stem nucleic acid, i.e. the second nucleic acid (effector nucleic acid) hybridizes only with the first nucleic acid (Env140) in the respective part of the stem nucleic acid of the first nucleic acid but not with other parts of the first nucleic acid (Env140). Thus, in order to increase the specificity/binding strength of the second nucleic acid additional complementary nucleotides outside of the first nucleic acid can be added.

The sequences herein are given in 5′-to-3′-direction according to the general convention for the presentation of nucleic acid sequences but it is evident for a person skilled in the art that the applied second nucleic acid will have reverse orientation in order to be complementary and hybridize with the first nucleic acid; thus the sequence complementary to a first nucleic acid presented in 5′-to-3′-direction has to be located at the 3′ end of a second nucleic acid presented in 5′-to-3′ direction and vice versa: alternatively present second nucleic acid of SEQ ID NO: 13 in 3′-to-5′-direction

An exemplary first nucleic acid according to the invention has been generated by replacing the first part of the stem nucleic acid sequence of Env140 by the sequence

(SEQ ID NO: 09)
CTG AGG GTA GT

and the corresponding sequence in the second part of the stem nucleic acid to be

(SEQ ID NO: 10)
ACT ACC CTC AG.

The effector nucleic acid hybridizes with the second part of the stem nucleic acid sequence and a further 7 nucleotides (shown in bold/highlighted in FIG. 7) outside of the Env140 sequence, i.e. in the backbone/poly A sequence.

For generating the respective stable cell line by double RMCE, respective front- and back-vectors have been designed (see FIG. 8). The RNA-module corresponds to the first nucleic acid according to the invention.

Restriction endonuclease sites at the 5′- and 3′-end served as linker and cloning sequences.

Two different stable cell lines have been generated:
cell line S69 with the LNA-regulatable sequence and constitutive activation:

(SEQ ID NO: 11)
CTG AGG GTA GTC CGG GGC UGG ACC GCC CCG CUG ACG AGG
CCC GCG GAG GGC CGA AAC TAC CCT CAG

cell line S70 with constitutive inactivation:

(SEQ ID NO: 12)
CTG AGG GTA GTC CGG GGC UGG ACC GCC CCG CUG ACG AGG
CCC GCG GAG GGC CGA GAC TAC CCT CAG

In FACS analysis, a clear difference with respect to GFP expression can be seen between the two cell lines and, thus, ribozyme forms (FIG. 9).

To the stable cell lines with the inducible/switchable construct as well as the inactive construct an LNA binding to the stem has been applied to modify the enzymatic cleavage. It can be seen that the fluorescence is shifted upon application of the LNA (FIG. 10).

LNA trigger sequence:

(SEQ ID NO: 13)
ATTGTGCCTGAGGGTAGT

Some nucleotides were used as locked nucleic acids in SEQ ID NO: 13, whereas every nucleotide of the oligo was synthesized with a phosphorothioate backbone.

12.5 μM LNA have been applied to the cultivation medium and median-fold change of fluorescence at 3 and 7 days after application to the cell line comprising the active/regulatable (S69) and the inactive (S70) ribozyme has been determined (FIG. 11).

These data perfectly show the working of the method and nucleic acid according to the invention.

Thus, the current invention encompasses at least the following independent aspects and dependent embodiments:
1. A first nucleic acid comprising in the following sequence
   a first part of a stem nucleic acid sequence,
   a cleavage site,
   a first stem loop nucleic acid sequences which 5′- and 3′-parts can form/form a duplex (loop I),
   a first part of a catalytic core sequence,
   a second stem loop nucleic acid sequences which 5′- and 3′-parts can form/form a duplex (loop II),
   a second part of the catalytic core sequence, and.
   a second part of the stem nucleic acid sequence, which is complementary to the first part of the stem nucleic acid sequence (and, thus, can form/forms a duplex therewith).
2. A first nucleic acid comprising in the following sequence
   a first part of a stem nucleic acid sequence,
   a cleavage site,
   a first stem loop nucleic acid sequences which 5′- and 3′-parts form a duplex denoted as loop I,
   a first part of a catalytic core sequence,
   a second stem loop nucleic acid sequences which 5′- and 3′-parts form a duplex denoted as loop II,
   a second part of the catalytic core sequence, and
   a second part of the stem nucleic acid sequence, which is complementary to the first part of the stem nucleic acid sequence and forms a duplex therewith.
3. The first nucleic acid according to any one of aspects 1 to 2, wherein the sequences are in 5′-to-3′-direction.
4. The first nucleic acid according to any one of aspects 1 to 2, wherein the sequences are in 3′-to-5′-direction.
5. A second nucleic acid, which is complementary to at least a part of the first or the second part of the stem nucleic acid sequence of the first nucleic acid according to any one of aspects 1 to 2 or any one of embodiments 3 to 4.
6. The second nucleic acid according to embodiment 5, wherein the binding of the second nucleic acid to the first nucleic acid results in a conformational change of the first nucleic acid.
7 The second nucleic acid according to any one of embodiments 5 to 6, wherein the binding of the second nucleic acid to the first nucleic acid results in a conformational change of the first nucleic acid, which is at least one of the following
   the dissociation of the first part of the first stem sequence from the second part of the stem sequence and the hybridization of one of the parts with the second nucleic acid,
   the dissociation of loop I and loop II resulting in an inactivation of the catalytic activity,
   the association of loop I and loop II resulting in an activation of the catalytic activity.
8. A composition comprising a pair of a first nucleic acid and a second nucleic acid,
   wherein the first nucleic acid comprises in the following sequence in 5′-to-3′-direction
      a first part of a stem nucleic acid sequence,
      a cleavage site,
      a first stem loop nucleic acid sequences which 5′- and 3′-parts form a duplex,
      a first part of a catalytic core sequence,
      a second stem loop nucleic acid sequences which 5′- and 3′-parts form a duplex,
      a second part of the catalytic core sequence, and
      a second part of the stem nucleic acid sequence, which is complementary to the first part of the stem nucleic acid sequence and forms a duplex therewith,
   wherein the second nucleic acid is complementary to at least a part of the first or the second part of the stem nucleic acid sequence and the part that is not complementary to the first or the second part of the stem nucleic acid is not complementary to another part of the first nucleic acid,
   wherein binding of the second nucleic acid to the first nucleic acid results in an inactivation of the catalytic activity of the first nucleic acid.
9. The composition according to aspect 8, wherein the binding of the second nucleic acid to the first nucleic acid results in a conformational change of the first nucleic acid, which is at least one of the following
   the dissociation of the first part of the first stem sequence from the second part of the stem sequence and the hybridization of one of the parts with the second nucleic acid,
   the dissociation of loop I and loop II resulting in an inactivation of the catalytic activity,
   the association of loop I and loop II resulting in an activation of the catalytic activity.
10. The first nucleic acid according to any one of aspects 1 to 2 or any one of embodiments 3 to 4 or the composition according to aspect 8 or embodiment 9, wherein the first nucleic acid is a hammerhead ribozyme and the stem is loop III of the hammerhead ribozyme.
11. The second nucleic acid according to aspect 5 or any one of embodiments 6, 7 or 10 or the composition according to aspect 8 or any one of embodiments 9 to 10, wherein the second nucleic acid is a LNA (locked nucleic acid).
12. A fusion nucleic acid comprising
   a coding nucleic acid encoding a selectable marker or a therapeutic protein or a regulatory compound,
   a nucleic acid comprising the first nucleic acid according to any one of aspects 1 or 2 or any one of embodiments 3, 4, 10 or 11, and
   a polyadenylation signal sequence that is operably linked to the coding nucleic acid.
13. The fusion nucleic acid according to aspect 12, wherein the coding nucleic acid is encoding a fluorescent protein or an antibody or a regulatory protein.
14. A composition comprising a fusion nucleic acid according to aspect 12 or embodiment 13 and a second nucleic acid that is complementary to at least a part of the first or the second part of the stem nucleic acid sequence of the first nucleic acid and the part that is not complementary to the first or the second part of the stem nucleic acid is not complementary to another part of the first nucleic acid.
15. The composition according to aspect 14, wherein the second nucleic acid is a second nucleic acid according to aspect 5 or any one of embodiments 6 to 7.
16. A mammalian cell comprising the first nucleic acid according to any one of aspects 1 or 2 or any one of embodiments 3, 4, 10 or 11 or a fusion nucleic acid according to aspect 12 or embodiment 13.
17. A method for selecting a pair of a first nucleic acid and a second nucleic acid according to aspect 8 or any one of embodiments 9, 10 or 11, comprising the following steps:
   providing a library of fusion nucleic acids according to aspect 12 or embodiment 13 wherein all or a part of the stem nucleic acid in the first nucleic acid is randomized,
   integrating the members of the library of the fusion nucleic acids into mammalian cells by targeted integration,
   single depositing the cells after the integration to generate a single cell library,
   determining the members of the single cell library in which the catalytic activity of the first nucleic acid can be changed by the addition of a corresponding library of second nucleic acids according to aspect 5 or any one of embodiments 6, 7 or 11,
   selecting a member of the single cell library wherein the difference of expression level of the coding nucleic acid in the absence and the presence of the second nucleic acid is larger than for other cells of the single cell library.
18. The method according to aspect 17, wherein the targeted integration is by recombinase mediated cassette exchange.
19. The method according to aspect 17 or embodiment 18, wherein the targeted integration is by double recombinase mediated cassette exchange.
20. The first nucleic acid according to any one of aspects 1 or 2 or any one of embodiments 3, 4, 10 or 11, wherein the first nucleic acid is within the 3′-UTR of a nucleic acid sequence encoding a selectable marker or a therapeutic protein or a regulatory compound.
21. The first nucleic acid according to embodiment 20, wherein the coding nucleic acid is encoding a fluorescent protein or an antibody or a regulatory protein.
22. The first nucleic acid according to any one of aspects 1 or 2 or any one of embodiments 3, 4, 10 or 11, wherein the first nucleic acid is after the coding part and before an operably linked polyA signal sequence of a nucleic acid sequence encoding a selectable marker or a therapeutic protein or a regulatory compound.
23. The first nucleic acid according to any one of aspects 1 or 2 or any one of embodiments 3, 4, 10, 11 or 22, wherein the coding sequence is 5′ to the first nucleic acid and the poly A signal sequence is 3′ to the first nucleic acid.
24. The first nucleic acid according to embodiment 22, wherein the coding nucleic acid is encoding a fluorescent protein or an antibody or a regulatory protein.
25. The fusion nucleic acid according to aspect 12 or embodiment 13 or the composition according to aspect 14 and embodiment 15 or the mammalian cell according to aspect 16 or the method according to aspect 17 or any one of embodiments 18 or 19 or the first nucleic acid according to any one of embodiments 20 to 24, wherein the coding sequence is in an expression cassette.
26. The second nucleic acid according to aspect 5 or any one of embodiments 6, 7 or 10 or the composition according to any one of aspect 8 or 14 or any one of embodiments 9, 10, 11 or 15 or the method according to aspect 17 or any one of embodiments 18 and 19, the second nucleic acid comprises a first part that is complementary to at least a part of the first or the second part of the stem nucleic acid sequence and a second part that is complementary to the nucleic acid sequence 5′ to the first part of the stem nucleic acid sequence in case the second nucleic acid is complementary to at least a part of the first part of the stem nucleic acid sequence or that is complementary to the nucleic acid sequence 3′ to the second part of the stem nucleic acid sequence in case the second nucleic acid is complementary to at least a part of the second part of the stem nucleic acid sequence.
27. The second nucleic acid according to aspect 5 or any one of embodiments 6, 7, 10 or 26 or the composition according to any one of aspect 8 or 14 or any one of embodiments 9, 10, 11, 15 or 26 or the method according to aspect 17 or any one of embodiments 18, 19 or 26, wherein the second nucleic acid is complementary to the second part of the stem nucleic acid sequence.
28. The second nucleic acid according to aspect 5 or any one of embodiments 6, 7, 10, 26 or 27 or the composition according to any one of aspect 8 or 14 or any one of embodiments 9, 10, 11, 15, 26 or 27 or the method according to aspect 17 or any one of embodiments 18, 19, 26 or 27, wherein the second nucleic acid comprises or consists of a first part that is complementary at least a part of the stem nucleic acid sequence and a second part that is not complementary to the first nucleic acid sequence.
29. The second nucleic acid according to aspect 5 or any one of embodiments 6, 7, 10, 26, 27 or 28 or the composition according to any one of aspect 8 or 14 or any one of embodiments 9, 10, 11, 15, 26, 27 or 28 or the method according to aspect 17 or any one of embodiments 18, 19, 26, 27 or 28, wherein the second nucleic acid is not complementary to the first nucleic acid outside the stem nucleic acid sequence,
30. The second nucleic acid according to aspect 5 or any one of embodiments 6, 7, 10, 26, 27, 28 or 29 or the composition according to any one of aspect 8 or 14 or any one of embodiments 9, 10, 11, 15, 26, 27, 28 or 29 or the method according to aspect 17 or any one of embodiments 18, 19, 26, 27, 28 or 29, wherein the second nucleic acid has a length of 8 to 21 nucleotides.
31. The second nucleic acid according to aspect 5 or any one of embodiments 6, 7, 10, 26, 27, 28 or 29 or the composition according to any one of aspect 8 or 14 or any one of embodiments 9, 10, 11, 15, 26, 27, 28 or 29 or the method according to aspect 17 or any one of embodiments 18, 19, 26, 27, 28 or 29, wherein the second nucleic acid has a length of 9 to 20 nucleotides.
32. The second nucleic acid according to aspect 5 or any one of embodiments 6, 7, 10, 26, 27, 28 or 29 or the composition according to any one of aspect 8 or 14 or any one of embodiments 9, 10, 11, 15, 26, 27, 28 or 29 or the method according to aspect 17 or any one of embodiments 18, 19, 26, 27, 28 or 29, wherein the second nucleic acid has a length of 11 to 18 nucleotides.
33. The second nucleic acid according to aspect 5 or any one of embodiments 6, 7, 10, 26, 27, 28, 29, 30, 31 or 32 or the composition according to any one of aspect 8 or 14 or any one of embodiments 9, 10, 11, 15, 26, 27, 28, 29, 30, 31 or 32 or the method according to aspect 17 or any one of embodiments 18, 19, 26, 27, 28, 29, 30, 31 or 32, wherein the second nucleic acid has a first part of 8-13 nucleotides that are complementary to the stem nucleic acid sequence of the first nucleic acid and a second part of 13 to 8 nucleotides that are complementary to a nucleic acid sequence 5′ or 3′ to the stem nucleic acid sequences of the first nucleic acid.
34. The second nucleic acid according to aspect 5 or any one of embodiments 6, 7, 10, 26, 27, 28, 29, 30, 31 or 32 or the composition according to any one of aspect 8 or 14 or any one of embodiments 9, 10, 11, 15, 26, 27, 28, 29, 30, 31 or 32 or the method according to aspect 17 or any one of embodiments 18, 19, 26, 27, 28, 29, 30, 31 or 32, wherein the second nucleic acid has a first part of about 11 nucleotides that are complementary to the stem nucleic acid sequence of the first nucleic acid and a second part of about 7 nucleotides that are complementary to a nucleic acid sequence 5′ or 3′ to the stem nucleic acid sequences of the first nucleic acid.
35. The second nucleic acid according to aspect 5 or any one of embodiments 6, 7, 10, 26, 27, 28, 29, 30, 31, 32, 33 or 34 or the composition according to any one of aspect 8 or 14 or any one of embodiments 9, 10, 11, 15, 26, 27, 28, 29, 30, 31, 32, 33 or 34 or the method according to aspect 17 or any one of embodiments 18, 19, 26, 27, 28, 29, 30, 31, 31, 33 or 34, wherein every nucleotide of the second nucleic acid has a phosphorothioate backbone.
36. The second nucleic acid according to aspect 5 or any one of embodiments 6, 7, 10, 26, 27, 28, 29, 30, 31, 32, 33, 34 or 35 or the composition according to any one of aspect 8 or 14 or any one of embodiments 9, 10, 11, 15, 26, 27, 28, 29, 30, 31, 32, 33, 34 or 35 or the method according to aspect 17 or any one of embodiments 18, 19, 26, 27, 28, 29, 30, 31, 31, 33, 34 or 35, wherein the second nucleic acid has the sequence of is ATT GTG CCT GAG GGT AGT (SEQ ID NO: 13).
37. The second nucleic acid according to aspect 5 or any one of embodiments 6, 7, 10, 26, 27, 28, 29, 30, 31, 32, 33, 34, 35 or 36 or the composition according to any one of aspect 8 or 14 or any one of embodiments 9, 10, 11, 15, 26, 27, 28, 29, 30, 31, 32, 33, 34, 35 or 36 or the method according to aspect 17 or any one of embodiments 18, 19, 26, 27, 28, 29, 30, 31, 31, 33, 34, 35 or 36, wherein the second nucleic acid comprises more nucleotides as those nucleotides complementary to the first or second part of the first nucleic acid, but which are not complementary to other parts of the first nucleic acid.
38. The first nucleic acid according to any one of aspects 1 or 2 or any one of embodiments 3, 4, 10, 11, 22, 23 or 24 or the composition according to any one of aspect 8 or 14 or any one of embodiments 9, 10, 11, 15, 26, 27, 28, 29, 30, 31, 32, 33, 34, 35, 36 or 37 or the method according to aspect 17 or any one of embodiments 18, 19, 26, 27, 28, 29, 30, 31, 31, 33, 34, 35, 36 or 37, wherein the first nucleic acid comprises
   as first part of a stem nucleic acid sequence the sequence of CCU CCU CGU (SEQ ID NO: 01),
   as cleavage site the sequence of CC (SEQ ID NO: 02),
   as first stem loop nucleic acid sequences, which 5′- and 3′-parts form a duplex, the sequence of GGG GCU GGA CCG CCC C (SEQ ID NO: 03),
   as first part of the catalytic core sequence the sequence of GCU GAC GA (SEQ ID NO: 04),
   as second stem loop nucleic acid sequences, which 5′- and 3′-parts form a duplex, the sequence of GGC CCG CGG AGG GCC (SEQ ID NO: 05),
   as second part of the catalytic core sequence the sequence of GAA (SEQ ID NO: 06),
   as second part of the stem nucleic acid sequence, which is complementary to the first part of the stem nucleic acid sequence, and forms a duplex therewith the sequence of ACG AGG AGG (SEQ ID NO: 08).
39. The first nucleic acid according to any one of aspects 1 or 2 or any one of embodiments 3, 4, 10, 11, 22, 23, 24 or 38 or the composition according to any one of aspect 8 or 14 or any one of embodiments 9, 10, 11, 15, 26, 27, 28, 29, 30, 31, 32, 33, 34, 35, 36, 37 or 38 or the method according to aspect 17 or any one of embodiments 18, 19, 26, 27, 28, 29, 30, 31, 31, 33, 34, 35, 36, 37 or 38, wherein the first part of the stem nucleic acid sequence has the sequence of CTG AGG GTA GT (SEQ ID NO: 09) and the second part of the stem nucleic acid sequence has the sequence of ACT ACC CTC AG (SEQ ID NO: 10).
40. Use of the first nucleic acid according to any one of aspects 1 or 2 or any one of embodiments 3, 4, 10, 11, 22, 23, 24, 38 or 39 or the composition according to any one of aspect 8 or 14 or any one of embodiments 9, 10, 11, 15, 26, 27, 28, 29, 30, 31, 32, 33, 34, 35, 36, 37, 38 or 39 for the selective activation or inactivation of the expression of an operably linked coding nucleic acid.
41. Use of the first nucleic acid according to any one of aspects 1 or 2 or any one of embodiments 3, 4, 10, 11, 22, 23, 24, 38 or 39 or the composition according to any one of aspect 8 or 14 or any one of embodiments 9, 10, 11, 15, 26, 27, 28, 29, 30, 31, 32, 33, 34, 35, 36, 37, 38 or 39 for the screening and selection of ribozyme-nucleic acid sequence pairs wherein the nucleic acid sequence activates or inhibits the catalytic activity of the ribozyme.
42. A method for selecting a variant ribozyme based on the leakiness or tightness of the regulating properties of the first nucleic acid according to any one of aspects 1 or 2 or any one of embodiments 3, 4, 10, 11, 22, 23, 24, 38 or 39 or the composition according to any one of aspect 8 or 14 or any one of embodiments 9, 10, 11, 15, 26, 27, 28, 29, 30, 31, 32, 33, 34, 35, 36, 37, 38 or 39.

The following examples, sequences and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Single nucleotide swap in the catalytic core (A→G) inactivates the self-cleaving activity of a hammerhead ribozyme: difference in GFP expression determined by FACS between active and inactive form of the ribozyme controlling GFP expression.

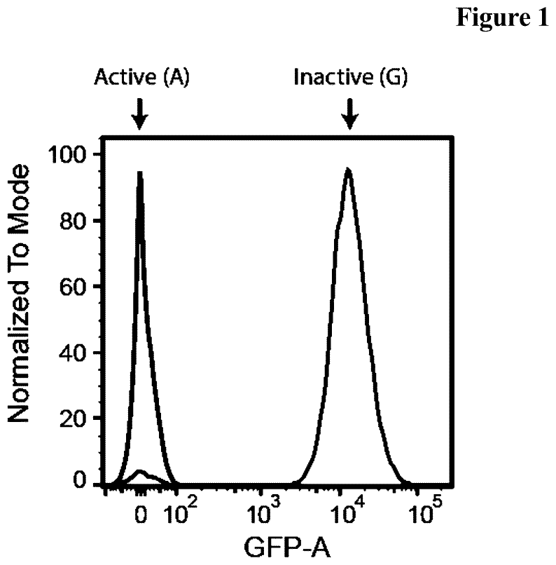

FIG. 2 Single nucleotide swap in the catalytic core (A→G) inactivates the self-cleaving activity of a hammerhead ribozyme: sketch of the construct.

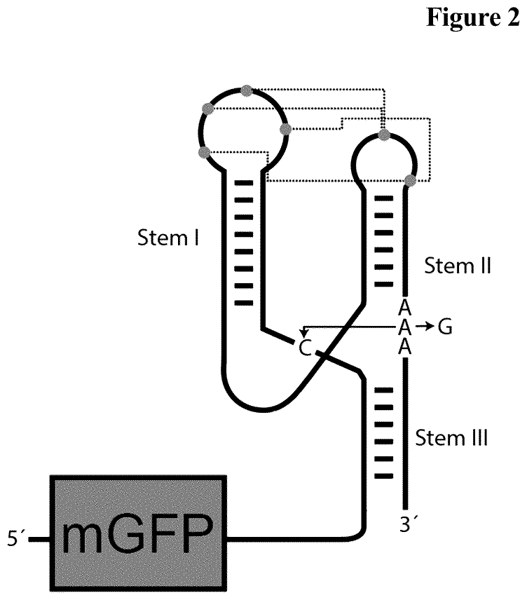

FIG. 3 Exemplification of the method according to the current invention for selecting variant ribozymes with minimal leakiness, i.e. expression, in the absence of an external stimulus, by selecting cells showing the highest expression of the marker gene in the presence of the stimulus and the lowest expression of the marker gene in the absence of the stimulus, and vice versa; the fold-inactivation or fold-activation change is used as a selection criterion.

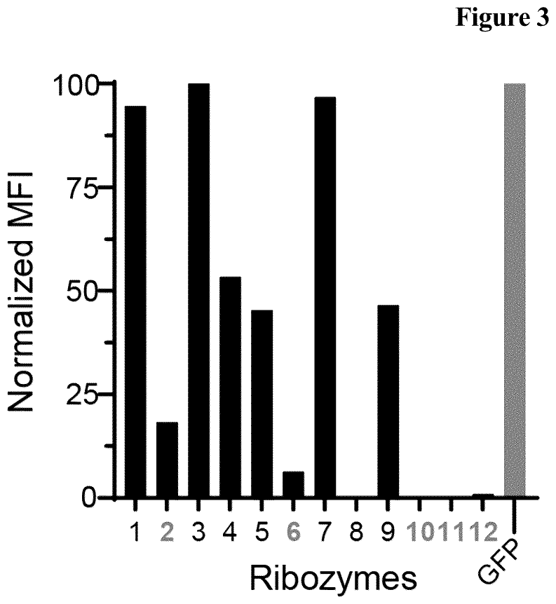

FIG. 4 Method according to the current invention exemplified using the K4/K19 tetracycline-responsive ribozyme-switch generated by Beilstein et al. (ACS Synth. Biol., 15 (2015) 526-534); although the output measurement was different (Luciferase vs GFP) a 1.9× fold-increase at 50 μM tetracycline concentration was achieved.

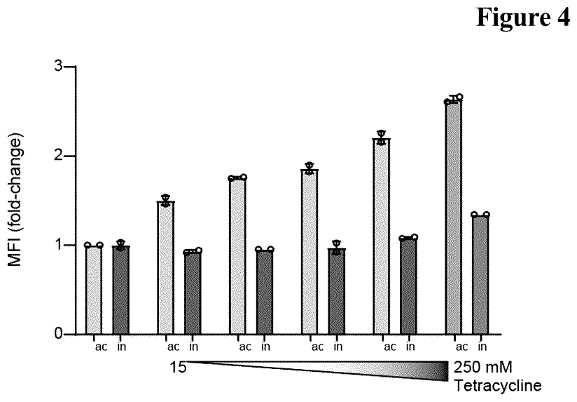

FIG. 5 Activation of GFP at 50 μM tetracycline concentration.

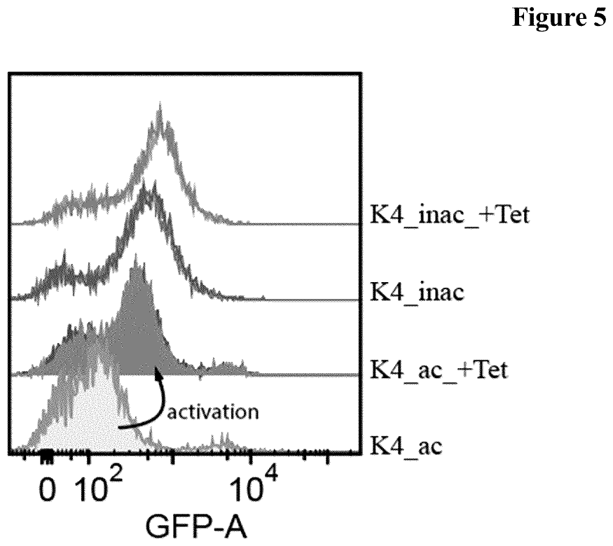

FIG. 6 Env140 as described by Auslaender et al. (Nucl. Acids Res., 44 (2016) e94) expressly incorporated herein by reference. Reproduction of FIG. 2A in combination with FIG. 3A of Auslaender et al.

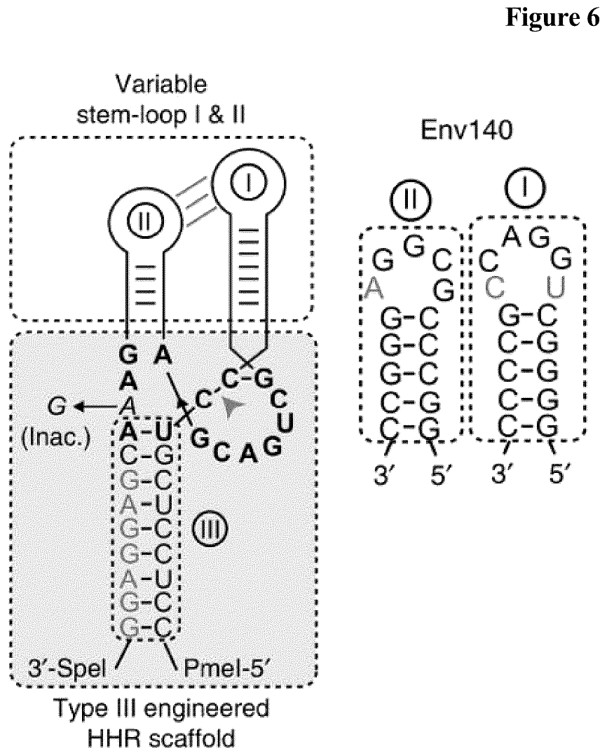

FIG. 7 Sketch of an exemplary first nucleic acid according to the invention based on Env140.

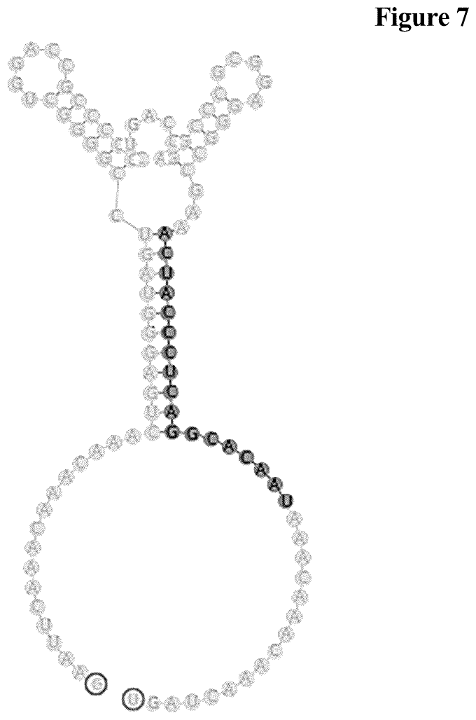

FIG. 8 Sketch of the front- and back-vectors designed for generating the respective stable cell line by double RMCE for stable expression of exemplary first nucleic acid according to the invention.

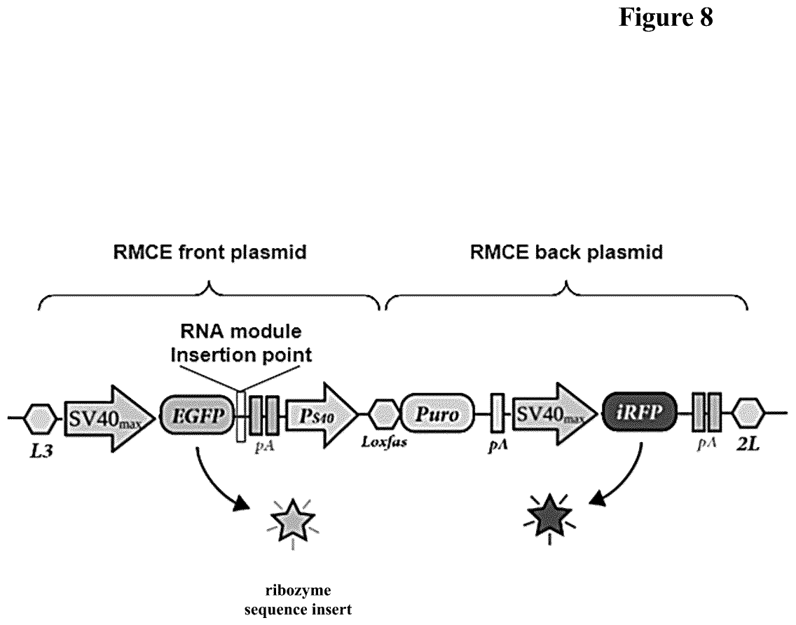

FIG. 9 In FACS analysis of GFP expression of the two cell lines and, thus, ribozyme forms.

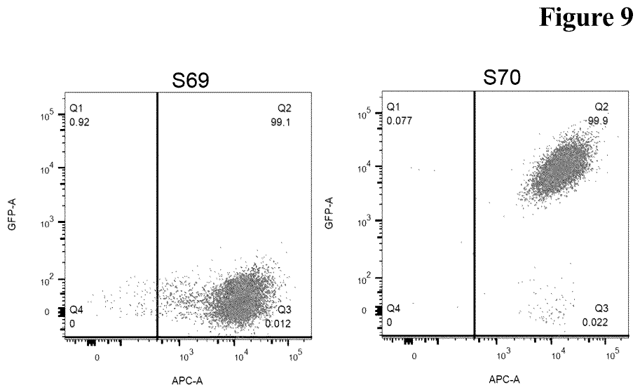

FIG. 10 To the stable cell lines with the inducible/switchable construct as well as the inactive construct an LNA binding to the stem has been applied to modify the enzymatic cleavage. It can be seen that the fluorescence is shifted upon application of the LNA

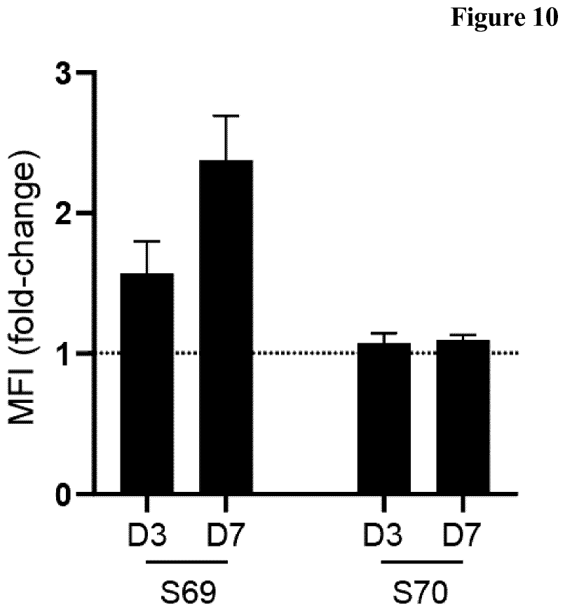

FIG. 11 Change of GFP expression after contact with 12.5 μM LNA; median-fold change of fluorescence at 3 and 7 days after application to the cell line comprising the active/regulatable (S69) and the inactive (S70) ribozyme.

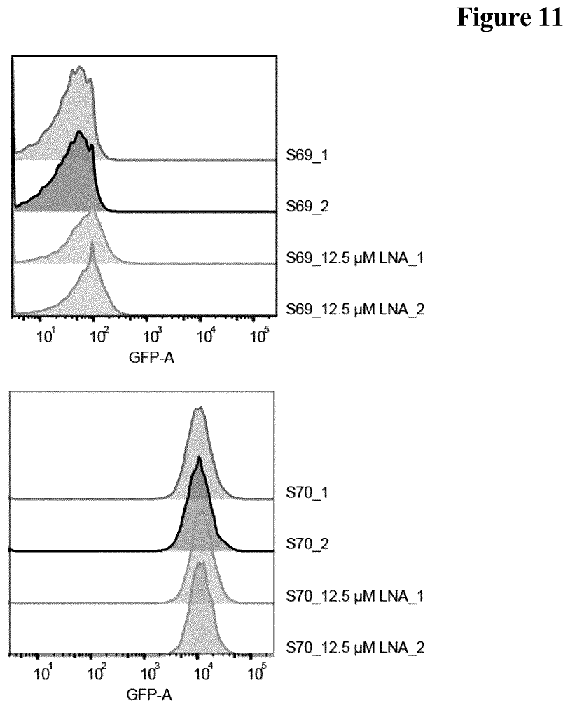

### DESCRIPTION OF THE EXAMPLES

### General

### Cell Culture

CHO TI-host cells (see WO 2019/126634) were cultured in a proprietary DMEM/F12-based medium at 37° C., and 5% CO₂ in 500 mL shake flask and were passaged every 3 to 4 days.

### Selection

Pool selection and maintenance was performed in 24 deep-well plate format using 4 mL culture volume and standard culture condition (37° C. 5% CO₂, 50 mm shaking amplitude at 225 rpm).

### Example 1

### Cloning and Pool Production

Targeted gene integration pool production followed the protocol established by Ng, D., et al: (Biotechnol. Prog. 37 (2021) e3140) with minor adjustments.

For testing and screening RNA-switches that control the mRNA degradation of a GFP reporter were cloned into the front plasmid, whereas the red-shifted iRFP gene was cloned into the back plasmid and was used for fluorescence normalization purposes. Expressed genes were placed under the control of a strong promoter (SV40). The RNA-switches constructs were cloned downstream of the GFP gene and before the bovine growth hormone polyadenylation (bGH-polyA) signal sequence. For each construct, both the complementary and anti-complementary strands were ordered as 5′-phosphorylated ssDNA oligonucleotides at Microsynth AG (Belgach, Switzerland) and were annealed by snap cooling at 0.166 μM concentration. The annealed construct contained overhangs for the direct ligation into the digested front plasmid, using EcoRI-EcoRI or EcoRI-SpeI recombination sites and EcoRI/SpeI-HF enzymes (NEB).

Production of targeted integration pools was performed using SF Cell Line 96-well Nucleofector™ Kit and 96-well Shuttle device (Lonza Group Ltd) according to the manufacturer's instructions. Briefly, 2 μg of front plasmid, 2 μg of back plasmid, and 0.8 μg Cre plasmid solution were added to a 20 μL solution containing about 2×10E6 host-TI cells resuspended in SF Cell Line Nucleofector-Supplement mix at a 4-5:1 ratio, according to manufacturer's protocol, and using the DS167 electroporation program. After electroporation, 80 μL warm culture medium was added to the electroporation chamber and incubated at 37° C. for 30 minutes, and pools were finally transferred in 24 well deep-well plate block for selection and maintenance.

Selection of double-integrated cells was performed after two days from electroporation by adding a concentrated solution of Puromycin (Life technologies, A11138-03) and FIAU (Sigma, #SML0632) to cultured cells, and selection was continued for 15-20 days until a >85% pool viability was reached, indicating the end of selection. Selected pools were maintained in media culture with half-amount of selection markers.

### Example 2

### LNA-Activation Assay and Flow Cytometry

To measure the LNA-triggered activation of GFP expression the stably-integrated pools expressing the active and inactive ribozyme construct. S69 and S70, were seeded at 50,000 cells/well into a flat-bottom round wells 96-well microplate, in 200 μL media, and incubated in standard static cell culture condition with or without 12.5 μM LNA oligonucleotide added from three to seven days, after which a 70 μL aliquot was removed and analyzed by flow cytometry. Flow cytometry was performed on a 4-6 color BD FACSCanto™ or FACS Celesta™ System using the FITC channel for the evaluation of GFP expression, and APC channel for iRFP expression. The population of cells was gated to avoid cell doublets detected by the forward and side scattering.

Sequence list

| name | sequence (5′-3′) |
|---|---|
| Env140_ac_fw | AATTCCTCCTCGTCCGGGGCTGGACCGCCCCGC |
| SEQ ID NO: 14 | TGACGAGGCCCGCGGAGGGCCGAAACGAGGAGG |
| Env140_ac_rv | CCTCCTCGTCCGGGGCTGGACCGCCCCGCTGAC |
| SEQ ID MNO: 15 | GAGGCCCGCGGAGGGCCGAAACGAGGAGGAATT |
| Env140_inac_fw | AATTCCTCCTCGTCCGGGGCTGGACCGCCCCGC |
| SEQ ID NO: 16 | TGACGAGGCCCGCGGAGGGCCGAGACGAGGAGG |
| Env140_inac_rv | AATTCCTCCTCGTCTCGGCCCTCCGCGGGCCTC |
| SEQ ID NO: 17 | GTCAGCGGGGCGGTCCAGCCCCGGACGAGGAGG |
| S69_ac_fw | AATTCAAACAAACAAACTGAGGGTAGTCCGGGG |
| SEQ ID NO: 18 | CTGGACCGCCCCGCTGACGAGGCCCGCGGAGGG |
| | CCGAAACTACCCTCAGGCACAATAAACAAACAA |
| | A |
| S69_ac_rv | CTAGTTTGTTTGTTTATTGTGCCTGAGGGTAGT |
| SEQ ID NO: 19 | TTCGGCCCTCCGCGGGCCTCGTCAGCGGGGCGG |
| | TCCAGCCCCGGACTACCCTCAGTTTGTTTGTTT |
| | G |
| S70_inac_fw | AATTCAAACAAACAAACTGAGGGTAGTCCGGGG |
| SEQ ID NO: 20 | CTGGACCGCCCCGCTGACGAGGCCCGCGGAGGG |
| | CCGAGACTACCCTCAGGCACAATAAACAAACAA |
| | A |
| S70_inac_rv | CTAGTTTGTTTGTTTATTGTGCCTGAGGGTAGT |
| SEQ ID NO: 21 | CTCGGCCCTCCGCGGGCCTCGTCAGCGGGGCGG |
| | TCCAGCCCCGGACTACCCTCAGTTTGTTTGTTT |
| | G |

1. A composition comprising a pair of a first nucleic acid and a second nucleic acid,
wherein the first nucleic acid comprises in the following sequence in 5′-to-3′-direction
a first part of a stem nucleic acid sequence,
a cleavage site,
a first stem loop nucleic acid sequences which 5′- and 3′-parts form a duplex,
a first part of a catalytic core sequence,
a second stem loop nucleic acid sequences which 5′- and 3′-parts form a duplex,
a second part of the catalytic core sequence, and
a second part of the stem nucleic acid sequence, which is complementary to the first part of the stem nucleic acid sequence and forms a duplex therewith,
wherein the second nucleic acid is complementary to at least a part of the first or the second part of the stem nucleic acid sequence and the part that is not complementary to the first or the second part of the stem nucleic acid is not complementary to another part of the first nucleic acid,
wherein binding of the second nucleic acid to the first nucleic acid results in an inactivation of the catalytic activity of the first nucleic acid.

2. The composition according to claim 1, wherein the binding of the second nucleic acid to the first nucleic acid results in a conformational change of the first nucleic acid, which is at least one of the following
the dissociation of the first part of the first stem sequence from the second part of the stem sequence and the hybridization of one of the parts with the second nucleic acid,
the dissociation of loop I and loop II resulting in an inactivation of the catalytic activity,
the association of loop I and loop II resulting in an activation of the catalytic activity.

3. The composition according to claim 1, wherein the first nucleic acid is a hammerhead ribozyme and the stem is loop III of the hammerhead ribozyme.

4. The composition according to claim 1, wherein the second nucleic acid is a LNA (locked nucleic acid).

5. A fusion nucleic acid comprising
a coding nucleic acid encoding a selectable marker or a therapeutic protein or a regulatory compound, in one preferred embodiment a fluorescent protein or an antibody or a regulatory protein,
a nucleic acid comprising the first nucleic acid according to claim 1, and
a polyadenylation signal sequence that is operably linked to the coding nucleic acid (in case the second nucleic acid sequence is catalytically inactive).

6. A mammalian cell comprising the first nucleic acid according to claim 1.

7. A method for selecting a pair of a first nucleic acid and a second nucleic acid comprising the following steps:
providing a library of fusion nucleic acids according to claim 5 wherein all or a part of the stem nucleic acid in the first nucleic acid according to the invention is randomized,
integrating the members of the fusion nucleic acid library into mammalian cells by targeted integration,
single depositing the cells after the integration to generate a single cell library,
determining the members of the single cell library in which the catalytic activity of the first nucleic acid can be changed by the addition of a corresponding second nucleic acid library,
selecting a member of the single cell library wherein the difference of expression level of the coding nucleic acid in the absence and the presence of the second nucleic acid is larger than for other cells of the single cell library.

8. The method according to claim 7, wherein the targeted integration is by recombinase mediated cassette exchange.

9. The method according to claim 7, wherein the targeted integration is by double recombinase mediated cassette exchange.

10. The composition of claim 2, wherein the first nucleic acid is a hammerhead ribozyme and the stem is loop III of the hammerhead ribozyme.

11. The composition of claim 2, wherein the second nucleic acid is a LNA (locked nucleic acid).

12. The composition of claim 3, wherein the second nucleic acid is a LNA (locked nucleic acid).

13. The composition of claim 10, wherein the second nucleic acid is a LNA (locked nucleic acid).

14. The fusion nucleic acid of claim 5, wherein binding of the second nucleic acid to the first nucleic acid results in a conformational change of the first nucleic acid, which is at least one of the following
the dissociation of the first part of the first stem sequence from the second part of the stem sequence and the hybridization of one of the parts with the second nucleic acid,
the dissociation of loop I and loop II resulting in an inactivation of the catalytic activity,
the association of loop I and loop II resulting in an activation of the catalytic activity.

15. The fusion nucleic acid of claim 5, wherein the first nucleic acid is a hammerhead ribozyme and the stem is loop III of the hammerhead ribozyme.

16. The fusion nucleic acid of claim 14, the first nucleic acid is a hammerhead ribozyme and the stem is loop III of the hammerhead ribozyme.


SEQUENCE LISTING (21 sequences)

SEQ ID NO: 1-2 - skipped (2 entries)

SEQ ID NO: 3 - RNA, 16 nt, synthetic construct
  misc_feature 1..16: note = first stem loop nucleic acid sequences
  source 1..16: mol_type = other RNA
ggggctggaccgcccc

SEQ ID NO: 4 - skipped

SEQ ID NO: 5 - RNA, 15 nt, synthetic construct
  misc_feature 1..15: note = second stem loop nucleic acid sequences
  source 1..15: mol_type = other RNA
ggcccgcggagggcc

SEQ ID NO: 6-8 - skipped (3 entries)

SEQ ID NO: 9 - DNA, 11 bp, synthetic construct
  misc_feature 1..11: note = first part of the stem
  source 1..11: mol_type = other DNA
ctgagggtagt

SEQ ID NO: 10 - DNA, 11 bp, synthetic construct
  misc_feature 1..11: note = second part of the stem
  source 1..11: mol_type = other DNA
actaccctcag

SEQ ID NO: 11 - DNA, 66 bp, synthetic construct
  misc_feature 1..66: note = S69 insert
  source 1..66: mol_type = other DNA
  modified_base 19: mod_base = OTHER; note = uracil
  modified_base 32: mod_base = OTHER; note = uracil
ctgagggtagtccggggctggaccgccccgctgacgaggcccgcggagggccgaaactaccctcag

SEQ ID NO: 12 - DNA, 66 bp, synthetic construct
  misc_feature 1..66: note = S70 insert
  source 1..66: mol_type = other DNA
  modified_base 19: mod_base = OTHER; note = uracil
  modified_base 32: mod_base = OTHER; note = uracil
ctgagggtagtccggggctggaccgccccgctgacgaggcccgcggagggccgagactaccctcag

SEQ ID NO: 13 - DNA, 18 bp, synthetic construct
  misc_feature 1..18: note = LNA trigger
  source 1..18: mol_type = other DNA
attgtgcctgagggtagt

SEQ ID NO: 14 - DNA, 66 bp, synthetic construct
  misc_feature 1..66: note = Env140_ac_fw
  source 1..66: mol_type = other DNA
aattcctcctcgtccggggctggaccgccccgctgacgaggcccgcggagggccgaaacgaggagg

SEQ ID NO: 15 - DNA, 66 bp, synthetic construct
  misc_feature 1..66: note = Env140_ac_rv
  source 1..66: mol_type = other DNA
cctcctcgtccggggctggaccgccccgctgacgaggcccgcggagggccgaaacgaggaggaatt

SEQ ID NO: 16 - DNA, 66 bp, synthetic construct
  misc_feature 1..66: note = Env140_inac_fw
  source 1..66: mol_type = other DNA
aattcctcctcgtccggggctggaccgccccgctgacgaggcccgcggagggccgagacgaggagg

SEQ ID NO: 17 - DNA, 66 bp, synthetic construct
  misc_feature 1..66: note = Env140_inac_rv
  source 1..66: mol_type = other DNA
aattcctcctcgtctcggccctccgcgggcctcgtcagcggggcggtccagccccggacgaggagg

SEQ ID NO: 18 - DNA, 100 bp, synthetic construct
  misc_feature 1..100: note = S69_ac_fw
  source 1..100: mol_type = other DNA
aattcaaacaaacaaactgagggtagtccggggctggaccgccccgctgacgaggcccgcggagggccgaaactaccctcaggcacaataaacaaacaaa

SEQ ID NO: 19 - DNA, 100 bp, synthetic construct
  misc_feature 1..100: note = S69_ac_rv
  source 1..100: mol_type = other DNA
ctagtttgtttgtttattgtgcctgagggtagtttcggccctccgcgggcctcgtcagcggggcggtccagccccggactaccctcagtttgtttgtttg

SEQ ID NO: 20 - DNA, 100 bp, synthetic construct
  misc_feature 1..100: note = S70_inac_fw
  source 1..100: mol_type = other DNA
aattcaaacaaacaaactgagggtagtccggggctggaccgccccgctgacgaggcccgcggagggccgagactaccctcaggcacaataaacaaacaaa

SEQ ID NO: 21 - DNA, 100 bp, synthetic construct
  misc_feature 1..100: note = S70_inac_rv
  source 1..100: mol_type = other DNA
ctagtttgtttgtttattgtgcctgagggtagtctcggccctccgcgggcctcgtcagcggggcggtccagccccggactaccctcagtttgtttgtttg